(19) United States
(12) Patent Application Publication
(10) Pub. No.: US 2012/0065267 A1
(43) Pub. Date: Mar. 15, 2012

(54) COMPOSITIONS INCLUDING 3,5-L-T2 AND METHODS OF USE THEREOF

(75) Inventors: Joel R.L. Ehrenkranz, Salt Lake City, UT (US); Thomas S. Scanlan, Portland, OR (US)

(73) Assignee: T*AMINE, LLC, Salt Lake City, UT (US)

(21) Appl. No.: 13/228,239

(22) Filed: Sep. 8, 2011

Related U.S. Application Data
(60) Provisional application No. 61/381,267, filed on Sep. 9, 2010.

(51) Int. Cl. A61K 31/195; A61P 3/06

(52) U.S. Cl. 514/567

(57) ABSTRACT

Compositions containing 3,5-L-T2 ((2S)-2-amino-3-[4-(4-hydroxyphenoxy)-3,5-diiodophenyl]propanoic acid) and methods for use thereof. Among other functions presented herein, 3,5-L-T2 can lower cholesterol via a mechanism that is independent of LDL receptor function. Disclosed compositions may include one or more additional active agents, such as, but not limited to, thyroid hormones other than 3,5-L-T2, cholesterol lowering agents, anti-diabetes agents, anti-hypertensives, vasodilators, inotropic agents, anti-coagulants, anti-anginals, anti-arrhythmics, leptin, leptin analogues, and adipokines, a vitamin and mineral composition. Disclosed methods include, but are not limited to, methods for treating hypercholesterolemia in a subject, and methods for treating at least one of metabolic syndrome, hypothyroidism, or thyroid suppression.

### CROSS-REFERENCE TO RELATED APPLICATIONS

This application claims the benefit of an priority to U.S. Provisional Application Ser. No. 61/381,267 filed 9 Sep. 2010 and entitled “COMPOSITIONS INCLUDING 3,5-L-T2 AND METHODS OF USE THEREOF,” the entirety of which is incorporated herein by reference.

### BACKGROUND

Thyroid hormone is an important regulator of vertebrate development and homeostasis. Thyroid hormone is critical for normal fetal brain development, and brain disorders such as cretinism can result from a lack of thyroid hormone in the developing fetus. In adults, thyroid hormone exerts effects in almost all tissues, and important processes such as metabolic rate, thermal regulation, lipid inventory, cardiac function, and bone maintenance are affected by thyroid hormone. Individuals with excess blood levels of thyroid hormone (hyperthyroid) generally have elevated metabolic rate and body temperature, decreased serum cholesterol, and increased heart rate compared to those with normal thyroid hormone levels (euthyroid). Conversely, hypothyroidism is characterized by depressed metabolic rate and body temperature, elevated serum cholesterol, and decreased heart rate compared to euthyroid controls.

Thyronines are generally regarded as the principle chemical form of thyroid hormone. Thyronines include two phenyl ring structures joined by an oxygen. The general structure of a thyronine ((2S)-2-amino-3-[4-(4-hydroxy-R₁,R₂-phenoxy)-R₃,R₄-phenyl]propanoic acid) can be seen at Formula 1:

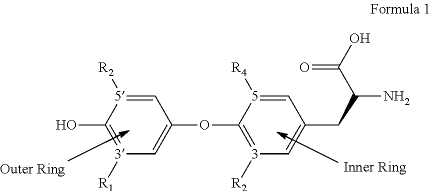

The two rings are referred to as the “inner ring” and the “outer ring.” As shown in Formula 1, the aminohydroxypropionic acid side chain on the inner ring includes a chiral center. Thyronines are produced in vivo by a series of enzymatically catalyzed reactions in the thyroid gland. Naturally occurring thyronines are typically derivatized with iodine at one or more of the 3 and 5 positions (R₃ and R₄) of the “inner ring” and the 3′ and 5′ positions (R₁ and R₂) of the “outer ring.” Naturally occurring thyronines are of the levorotatory or L form; the chiral center associated with the propanoic acid moiety has an absolute stereochemistry of S.

3,5,3′,5′-tetra-iodo-L-thyronine (“Thyroxine,” “T4,” or (2S)-2-amino-3-[4-(4-hydroxy-3,5-diiodophenoxy)-3,5-diiodophenyl]propanoic acid) is the predominant form of thyroid hormone that is secreted from the thyroid gland. T4 is synthesized by enzymes in the thyroid by joining the phenyl rings of two tyrosine residues and iodinating the two phenyl ring with a total of four iodine atoms at the R₁-R₄ positions of Formula 1. The structure of T4 is shown below at Formula 1A:

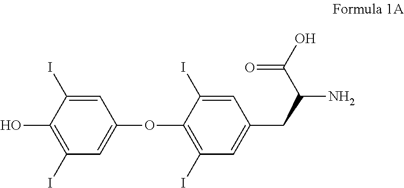

T4 is not the active form of thyroid hormone. Instead, T4 is converted to the physiologically active 3,5,3′-triiodo-L-thyronine (“T3,” (2S)-2-amino-3-[4-(4-hydroxy-3-iodophenoxy)-3,5-diiodophenyl]propanoic acid) by enzymatic deiodination in peripheral target tissues. T3 is shown below at Formula 1B:

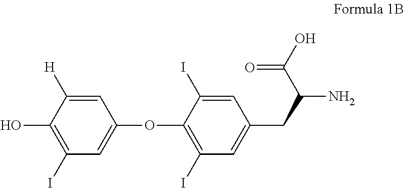

Three different deiodinases have been identified to date (D-I, D-II, and D-III). The D-I and D-II enzymes mediate “outer ring” deiodination such as the conversion of T4 to T3. In contrast, the D-III enzyme mediates “inner ring” deiodination, exemplified by the conversion of T4 to 3,3′,5′-triiodo-L-thyronine (“reverse-T3” or “rT3”). rT3 is shown below at Formula 1C:

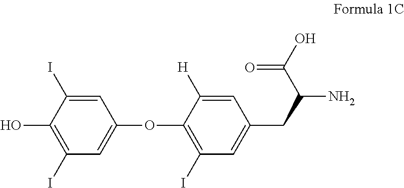

To date, no significant biological activity has been ascribed to rT3 even though significant blood levels of this metabolite are found. A variety of further deiodinated and lesser iodinated thyronines are known to exist in vivo. For example, (2S)-2-amino-3-[4-(4-hydroxyphenoxy)-3,5-diiodophenyl]propanoic acid (“3,5-L-T2” or “T2”) may be made directly in the thyroid or it may be made by diodination of T4 or T3. 3,5-L-T2 is shown below at Formula 1D:

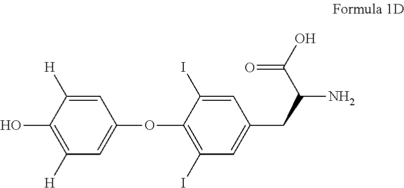

The first report showing a biological activity for T2 appeared in 1927. The effects of T2 are qualitatively distinct from those of T3. Specifically, T2 does not affect the pituitary thyroid axis, has selective effects on liver and brown fat, and demonstrates an onset of action, time to maximal effect, and duration of action significantly shorter than that of T3. T2's effects on mitochondrial energy production are not disrupted by protein synthesis inhibitors such as cyclophosphamide and actinomicin D, both of which completely block the mitochondrial effects of T3. In vitro studies show that T3 binds to TR α1, β1 and β2 with an affinity 40-500 fold greater than that of T2. T2, in contrast to T3, has no effect on thyroid receptor homodimer formation, is 50% less potent than T3 in decreasing TR β gene expression and 1/100 as potent as T3 in increasing growth hormone gene expression. T2 has <1% the potency of T3 in an in vivo anti-goiter assay and lacks central thyromimetic activity.

Immunoassay measurement of circulating total T2 levels in humans shows a concentration range of 0.4 to 10 ng/dl. There are no data on protein binding of T2 in serum. T2 levels decline with age and are higher in men than women. Women, however, produce more T2 and clear T2 more rapidly than men. T2 levels are increased in hyperthyroidism and decreased in hypothyroidism and sepsis.

A number of investigations from 1933 to the present have demonstrated T2's effects. For instance, T2 has been shown to have an effect on resting metabolic rate. The mechanism by which T2 increases metabolic rate is thought to be mediated by T2 acting on mitochondrial energy production. In vitro and in vivo studies have demonstrated that mitochondria have specific T2 binding sites. T2 increases mitochondrial cytochrome c oxidase, fatty acid and triacylglycerol synthesis, lipid oxidation, importing of fatty acids, F₀F₁ ATP synthase, and activates the AMPK-ACC-malonyl CoA pathway. In addition, chronic T2 administration to hypothyroid rats improves cold tolerance and normalizes somatic growth rates. In another study, it was found in rats that T2 administration (250 mcg/kg p.o. q.d. for 30 days) could decrease body weight by 13%, increase hepatic fat oxidation by 42%, decrease hepatic fat mass by 50%, and lower serum triglycerides by 52% and cholesterol by 18%, compared to controls. These metabolic effects were not accompanied by an increase in heart rate, altered thyroid gland or heart weight, changes in circulating TSH, free T3 or free T4 levels, or a blunted TSH response to TRH.

In another example, in 1960 McClure, de Mowbray, and Gilland administered a daily dose of 300 mg 3,5-D-T2 (i.e., the non-physiological stereoisomer of T2) for 8 months to 20 hypercholesterolemic patients, 13 of whom had coronary artery disease and 10 of whom had a history of myocardial infarction or angina. Of the remaining 20, 13 had no known atherosclerosis and 4 suffered from myxedema. McClure et al. observed a 5% decrease in body weight in euthyroid subjects and an 8% decrease in hypothyroid patients. Total serum cholesterol decreased by 20% at 20 weeks. An increase in mean heart rate from 76 to 88 beats per minute occurred. 7 of 13 patients with coronary artery disease experienced increased anginal symptoms. 2 of the 13 died suddenly from a presumed myocardial infarction. McClure et al. found that doses of less than 250 mg/day were ineffective and that patients receiving such a low dose had a tendency to “escape.” A daily dose of 300 mg/day 3,5-D-T2 represents a 100 fold excess over T2 doses subsequently shown to have maximal effects on mitochondrial energy production.

Another class of thyroid hormones, known as thyronamines, are thought to be produced by decarboxylation of thyronines. The enzymatic pathway responsible for decarboxylating thyronines is presently unknown, although it is postulated that the aromatic amino acid decarboxylase that normally produces dopamine and serotonin could also act on iodothyronines. It is also believed that decarboxylases in the stomach may be able to decarboxylate thyronines that are consumed as part of the diet in order to produce thyronamines. A general thyronamine can be described by Formula 2, shown below:

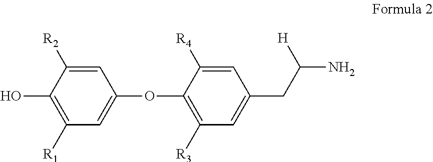

Thyronamines are similar to thyronines, except that the carboxyl group attached to the inner ring alkyl group is removed and replaced by a hydrogen. As shown in Formula 2, the chiral center is lost when a thyronine is decarboxylated to form a thyronamine. Thyronamines are similar to thyronines in that R₁-R₄ positions can be occupied by either iodine or hydrogen.

An example of a thyronamine is 3-iodothyronamine (“T1AM”), which is shown below at Formula 2A:

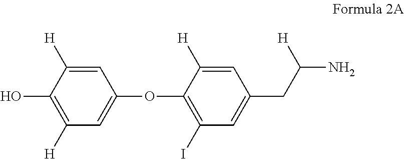

T1AM has been shown to be an endogenous component of biogenic amine extracts from rodent brain, liver, heart and blood.

### BRIEF SUMMARY

Obesity, hyperlipidemia, hypercholesterolemia, and other unhealthy lifestyle choices represent major risk factors for diabetes, heart disease, stroke, and cancer. Interventions such as diet, exercise, surgical procedures, and medications which produce weight loss or lower cholesterol decrease the incidence of these major causes of morbidity and mortality. Compliance with lifestyle changes, such as diet and exercise, is very difficult to maintain, bariatric surgery is invasive, and medications that lower cholesterol are weight neutral and require a physician's prescription. 3,5-L-T2 ((2S)-2-amino-3-[4-(4-hydroxyphenoxy)-3,5-diiodophenyl]propanoic acid) (“3,5-L-T2” or “T2”) may represent a novel agent with a unique mechanism of action for intervening in the pathophysiology of these disorders.

In one embodiment, a composition is disclosed. The composition includes a first active agent comprising 3,5-L-T2 ((2S)-2-amino-3-[4-(4-hydroxyphenoxy)-3,5-diiodophenyl]propanoic acid)

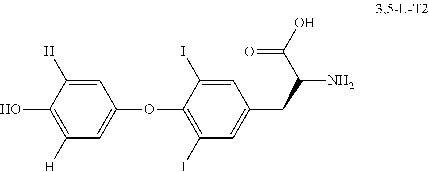

or a prodrug, pharmaceutically acceptable salt, hydrate, solvate, acid salt hydrate, N-oxide, or isomorphic crystalline salt thereof. The composition may further include a second active agent selected from the group consisting of T4, T3, a cholesterol lowering agent, an anti-diabetes agent, an anti-hypertensive, an anti-coagulant, an anti-anginal, an anti-arrhythmic, a vitamin and mineral composition, and combinations thereof.

In one embodiment, the composition can further include a third active agent that can be co-administered with the first and second active agent. The third active agent is selected from the group consisting of T4AM, rT3, rT3AM, 3,3′-T2, 3,3′-T2AM, 3,5-T2AM, T1, T1AM, T0, T0AM, and combinations thereof or a prodrug, pharmaceutically acceptable salt, hydrate, solvate, acid salt hydrate, N-oxide, or isomorphic crystalline salt thereof.

In another embodiment, a method for treating hypercholesterolemia in a subject is disclosed. The method includes (1) identifying a subject having an elevated serum cholesterol level, (2) administering to the subject a daily dosage between about 1 mcg and about 5000 mg of 3,5-L-T2 or a prodrug, pharmaceutically acceptable salt, hydrate, solvate, acid salt hydrate, N-oxide, or isomorphic crystalline salt thereof, and (3) obtaining an effect of lowering the subject's serum cholesterol level.

In one embodiment, the subject is a human. In another embodiment, the subject may be incompletely responsive to statin treatment or otherwise unsuited to statin treatment.

In one embodiment, a method for treating at least one of metabolic syndrome, hypothyroidism, or thyroid suppression is disclosed. The method includes (1) administering to a human a daily dosage of between about 1 mcg and about 5000 mg of 3,5-L-T2 or a prodrug, pharmaceutically acceptable salt, hydrate, solvate, acid salt hydrate, N-oxide, or isomorphic crystalline salt thereof, and (2) obtaining an effect of establishing or maintaining a healthy metabolism and/or establishing or maintaining healthy endocrine function.

In one embodiment, the daily dosage of 3,5-L-T2 or a prodrug, pharmaceutically acceptable salt, hydrate, solvate, acid salt hydrate, N-oxide, or isomorphic crystalline salt thereof includes a low dose of about 0.2-0.3 mg/kg of body weight to a high dose of about 2-3 mg/kg of body weight.

In one embodiment, a method for treating at least one of metabolic syndrome, hypothyroidism, or thyroid suppression may further include co-administering an effective amount of one or more of T4, T3, T4AM, rT3, rT3AM, 3,3′-T2, 3,3′-T2AM, 3,5-T2AM, T1, T1AM, T0, and T0AM, wherein the effective amount comprises a daily dosage of between about 1 mcg and about 5000 mg. In one embodiment, the daily dosage is administered in a fortified food or beverage composition.

These and other objects and features of the present invention will become more fully apparent from the following description and appended claims, or may be learned by the practice of the invention as set forth hereinafter.

### DETAILED DESCRIPTION

### I. Introduction and Background

Obesity, hyperlipidemia, hypercholesterolemia, and other unhealthy lifestyle choices represent major risk factors for diabetes, heart disease, stroke, and cancer. Interventions such as diet, exercise, surgical procedures, and medications which produce weight loss or lower cholesterol decrease the incidence of these major causes of morbidity and mortality. Compliance with lifestyle changes, such as diet and exercise, is very difficult to maintain, bariatric surgery is invasive, and medications that lower cholesterol are weight neutral and require a physician's prescription. 3,5-L-T2 ((2S)-2-amino-3-[4-(4-hydroxyphenoxy)-3,5-diiodophenyl]propanoic acid) (“3,5-L-T2” or “T2”) may represent a novel agent with a unique mechanism of action for intervening in the pathophysiology of these disorders.

3,5-L-T2 is a naturally occurring thyroid hormone known to affect hepatic and skeletal muscle oxidative metabolism and resting metabolic rate in euthyroid animals without affecting the pituitary thyroid axis. 3,5-L-T2 appears to have the potential for safely and effectively mitigating the effects of increased carbohydrate and/or fat intake on normal human metabolic activity without disrupting endogenous thyroid function.

The embodiments illustrated herein are based partly on the surprising and unexpected discovery that intake of 3,5-L-T2 is more effective than well-known statin drugs at lowering cholesterol and that, even more surprisingly, 3,5-L-T2 can decrease cholesterol via a mechanism that is independent of the low-density lipoprotein receptor (LDLr). In addition, the inventors have discovered that intake of 3,5-L-T2 can lower blood sugar levels relative to controls, which suggests that 3,5-L-T2 may be an effective diabetes treatment. Moreover, 3,5-L-T2 may be combined with other thyroid hormones (e.g., T4 or T3), cholesterol lowering agents (e.g., statins), anti-diabetes agents, anti-hypertensives, anti-coagulants, anti-anginals, anti-arrhythmics, and/or vitamin and mineral compositions in order to augment the effects of known and yet to be discovered therapeutics and to help maintain healthy triglyceride and cholesterol levels, healthy weight, cardiovascular health, healthy sleep patterns, healthy mood, healthy skin, healthy nails, healthy endocrine function, healthy metabolism, healthy neuropsychiatric function, and a number of other markers of associated with general health and well-being.

Metabolic abnormalities such as, but not limited to, diabetes, hyperglycemia, hypothyroidism, and metabolic syndrome are often related—the factors relating these disorders may include, but are not limited to, reduced metabolic rate, high blood sugar, high body mass, elevated triglycerides, and the like. 0.55% of the US population and approximately 10% of postmenopausal women are hypothyroid. Symptoms of hypothyroidism include poor muscle tone (muscle hypotonia), elevated serum cholesterol, cold intolerance, depression, weight gain, and low heart rate. types I and II diabetes are often associated obesity and chronic hyperglycemia (i.e., elevated blood sugar); in addition, type II diabetes is generally associated with insulin resistance. Insulin resistance refers to the diminished ability of cells to respond to the action of insulin in promoting the transport of the sugar glucose, from blood into muscles and other tissues. The main features of metabolic syndrome include insulin resistance, hypertension (high blood pressure), cholesterol abnormalities, and an increased risk for clotting. Patients diagnosed with metabolic syndrome are most often overweight or obese.

There are a number of working definitions of metabolic syndrome depending on which group of experts is doing the defining. For example, based on the guidelines from the 2001 National Cholesterol Education Program Adult Treatment Panel (ATP III), any three of the following traits in the same individual meet the criteria for the metabolic syndrome:

1. Abdominal obesity: a waist circumference over 102 cm (40 in) in men and over 88 cm (35 inches) in women.

2. Serum triglycerides 150 mg/dl or above.

3. HDL cholesterol 40 mg/dl or lower in men and 50 mg/dl or lower in women.

4. Blood pressure of 130/85 or more.

5. Fasting blood glucose of 110 mg/dl or above. (Some groups say 100 mg/dl) The World Health Organization (WHO) has slightly different criteria for the metabolic syndrome:

1. High insulin levels, an elevated fasting blood glucose or an elevated post meal glucose alone with at least 2 of the following criteria:

2. Abdominal obesity as defined by a waist to hip ratio of greater than 0.9, a body mass index of at least 30 kg/m² or a waist measurement over 37 inches.

3. Cholesterol panel showing a triglyceride level of at least 150 mg/dl or an HDL cholesterol lower than 35 mg/dl.

4. Blood pressure of 140/90 or above (or on treatment for high blood pressure).

Chronic metabolic abnormalities, including obesity, dyslipidemia, metabolic syndrome, and hypothyroidism, are widespread. They represent the root cause of a number of diseases associated with substantial medical complications and health care expenses and are challenging to correct. New therapeutic strategies for treating obesity or lipid abnormalities have not been introduced in this decade nor have approaches for studying the chronic complaints commonly encountered in treated hypothyroidism been developed.

Although the treatment of hypothyroidism with thyroxine is well accepted and generally effective in normalizing circulating TSH and free T4 levels, a significant number of hypothyroid patients receiving thyroxine (i.e., T4) report persistent subjective complaints despite standard therapy. The physiological basis for this incomplete treatment response is unclear.

Adjuvant treatment of hypothyroidism with supplemental T3 has received attention, and the interest generated by research in this area demonstrates the widespread prevalence and clinical significance of this problem. Circulating T2 levels are also reported to be low in hypothyroid patients, but T4 and T3 are not thought to be converted to T2 in vivo. Anecdotal observations suggest that T2 has beneficial effects in myxedema (i.e., a disease resulting from the decreased function of the thyroid, characterized by a slowing down of mental and physical activity and thickening and drying of the skin). The role of T2 in the pathogenesis and treatment of hypothyroidism is an area which requires additional investigation.

There may be a number of situations where it may be advantageous to administer T2 to subjects having thyroid related conditions. In one example, it is often observed that individuals receiving T4 and/or T4 and T3 therapy treatment of hypothyroidism will continue to display symptoms of having reduced thyroid hormone in spite of the fact that their T4/T3 levels appear normal. It is believed that augmenting T2 levels in these patients may ameliorate some of these side effects without having to overdose patients on T4. T4 and T3 can also be given to patients with conditions such as thyroid cancer and non-toxic goiter to suppress the thyroid. It is believed that augmenting T2 levels in these patients may be effective for further suppressing the thyroid gland without having to overdose patients on T4 or T3. In addition, as discussed in greater detail above, T2 may be effective for treating high cholesterol, diabetes, metabolic syndrome, and a number of related disorders.

### II. Compositions and Methods

In one embodiment, a composition is disclosed. The composition includes a first active agent comprising 3,5-L-T2 ((2S)-2-amino-3-[4-(4-hydroxyphenoxy)-3,5-diiodophenyl]propanoic acid)

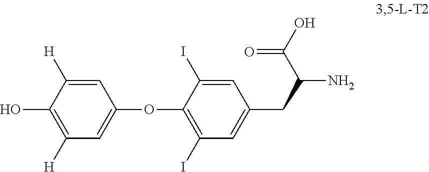

or a prodrug, pharmaceutically acceptable salt, hydrate, solvate, acid salt hydrate, N-oxide, or isomorphic crystalline salt thereof. The composition further includes a second active agent selected from the group consisting of T4, T3, a cholesterol lowering agent, an anti-diabetes agent, an anti-hypertensive, an anti-coagulant, an anti-anginal, an anti-arrhythmic, a vitamin and mineral composition, and combinations thereof.

In one embodiment, the composition can further include a third active agent that can be co-administered with the first and second active agent. The third active agent is selected from the group consisting of T4AM, rT3, rT3AM, 3,3′-T2, 3,3′-T2AM, 3,5-T2AM, T1, T1AM, T0, T0AM, and combinations thereof or a prodrug, pharmaceutically acceptable salt, hydrate, solvate, acid salt hydrate, N-oxide, or isomorphic crystalline salt thereof.

Suitable examples of cholesterol lowering agents include, but are not limited to, statins (e.g., atorvastatin), bile acid sequestrants (e.g., cholestyramine or colestipol), nicotinic acid preparations, fibrates (e.g., bezafibrate, ciprofibrate, clofibrate, gemfibrozil, and fenofibrate), and compounds that inhibit digestive absorption of cholesterol (ezetimibe), and combinations thereof.

Suitable examples of anti-diabetes agents include, but are not limited to, insulin, sulfonylureas, meglitinides, biguanides, thiazolidinediones, alpha-glucosidase inhibitors (miglitol (Glyset™), acarbose (Precose™/Glucobay™), peptide analogs (Incretin mimetics, glucagon-like peptide (GLP) analogs and agonists, DPP-4 inhibitors, amylin analogues), and combinations thereof.

Suitable examples of anti-hypertensive agents include, but are not limited to, ACE inhibitors (e.g., captopril), angiotensin II receptor antagonists (e.g., losartan), alpha blockers (e.g., doxazosin), beta blockers (e.g., propranolol), mixed alpha+beta blockers (e.g., bucindolol), calcium channel blockers (e.g., verapamil), aldosterone receptor antagonists (e.g., eplerenone and spironolactone), vasodilators, diuretics (e.g. hydrochlorothiazide), direct renin inhibitors (e.g., aliskiren), and combinations thereof.

Suitable examples of anti-coagulants include, but are not limited to, coumadins, heparins, direct thrombin inhibitors (e.g., argatroban, lepirudin, bivalirudin, and dabigatran), antiplatelet agents (e.g., Plavix™, aka clopidogrel), aspirin, and combinations thereof.

Suitable examples of anti-anginals include, but are not limited to, nitrates, beta blockers, calcium channel blockers, and combinations thereof.

Suitable examples of anti-arrhythmics include, but are not limited to sodium channel blockers (e.g., quinidine, procainamide, disopyramide, lidocaine, phenyloin, mexiletine), beta blockers (e.g., propranolol, esmolol, timolol, metoprolol, atenolol, bisoprolol), potassium channel blockers (e.g., amiodarone, sotalol, ibutilide, dofetilide, and E-4031), calcium channel blockers (e.g., verapamil, diltiazem), adenosine, digoxin, and combinations thereof.

Suitable examples of compounds that can be included in a vitamin and mineral composition include, but are not limited to, vitamins A, B₁, B₂, B₃, B₅, B₆, B₇, B₉, B₁₂, C, D, E, and K and/or one or more of potassium, chlorine, sodium, calcium, phosphorus, magnesium, zinc, iron, manganese, copper, iodine, selenium, chromium, molybdenum, and combinations thereof.

In another embodiment, a method for treating at least one of diabetes mellitus, hyperglycemia, or metabolic syndrome is disclosed. The method includes (1) administering to a human a daily dosage of between about 1 mcg and about 5000 mg of 3,5-L-T2 or a prodrug, pharmaceutically acceptable salt, hydrate, solvate, acid salt hydrate, N-oxide, or isomorphic crystalline salt thereof, and (2) obtaining an effect of establishing or maintaining at least one of a healthy blood sugar level, a healthy weight, a healthy insulin level, a healthy cholesterol level, or a healthy blood pressure.

In yet another embodiment, a method for treating hypercholesterolemia in a subject is disclosed. The method includes (1) identifying a subject having an elevated serum cholesterol level, (2) administering to the subject a daily dosage between about 1 mcg and about 5000 mg of 3,5-L-T2 or a prodrug, pharmaceutically acceptable salt, hydrate, solvate, acid salt hydrate, N-oxide, or isomorphic crystalline salt thereof, and (3) obtaining an effect of lowering the subject's serum cholesterol level.

In one embodiment, the subject is a human. In another embodiment, the subject may be incompletely responsive to statin treatment or otherwise unsuited to statin treatment.

Statin drugs reduce serum cholesterol levels in patients by a number of mechanisms. First, statins act by competitively inhibiting HMG-CoA reductase, the first committed enzyme of the cholesterol biosynthesis pathway. This reduces the amount of cholesterol produced in the liver and peripheral tissues. This is significant because most circulating cholesterol comes from internal manufacture rather than the diet.

Inhibition of cholesterol biosynthesis by statins also stimulates the production of low-density lipoprotein receptor (“LDLr”) molecules. LDLr is responsible for collecting LDL-bound cholesterol in the blood and transporting it into cells. Statin treatment is ineffective for reducing serum cholesterol levels in patients that do not possess at least a threshold level (e.g., about 10% of wild-type) of LDLr function. As such, patients who do not have at least a threshold level of LDLr function will not respond to statin treatment.

Surprisingly and unexpectedly, the inventors in this case have found that T2 may be more effective than statins at reducing serum cholesterol levels and that T2 is capable of lowering cholesterol via an as yet unknown mechanism that is independent of LDLr. Thus, T2 represents a novel cholesterol lowering treatment for patients that are not responsive to statin drugs.

In addition, statin drugs are known to have a number of potentially serious side-effects. For example, side-effects associated with statin treatment include, but are not limited to, muscle pain, muscle pain, muscle weakness, muscle tenderness, myositis, myopathy, rhabdomyolysis, neuropathy, memory loss, changes in liver function, liver failure, changes in kidney function, kidney failure, and combinations thereof. Many of these side-effects are potentially life threatening (e.g., rhabdomyolysis and liver failure) and patients suffering from such side-effects generally have to immediately cease statin treatment. Thus, T2 represents a novel cholesterol lowering treatment for patients that are unable, for one reason or another, to take statin drugs.

In one embodiment, the daily dosage may be administered in a fortified food or beverage composition. Suitable examples of fortified foods or beverage compositions include, but are not limited to, processed meat products, processed fish products, gels, jams, pastes, nutrition bars, bakery products, creams, sauces, dairy products, confections, syrups, pet foods, water-based beverages, or dairy-based beverages, combinations thereof, and the like.

In one embodiment, the fortified food or beverage composition includes about 0.01 wt % to about 99.9 wt %, about 0.1 wt % to about 60 wt %, or about 1 wt % to about 50 wt % of 3,5-L-T2 or a prodrug, pharmaceutically acceptable salt, hydrate, solvate, acid salt hydrate, N-oxide, or isomorphic crystalline salt thereof.

In one embodiment, a method for treating at least one of metabolic syndrome, hypothyroidism, or thyroid suppression is disclosed. The method includes (1) administering to a human a daily dosage of between about 1 mcg and about 5000 mg of 3,5-L-T2 or a prodrug, pharmaceutically acceptable salt, hydrate, solvate, acid salt hydrate, N-oxide, or isomorphic crystalline salt thereof, and (2) obtaining an effect of establishing or maintaining a healthy metabolism and/or establishing or maintaining healthy endocrine function.

In one embodiment, the daily dosage of 3,5-L-T2 or a prodrug, pharmaceutically acceptable salt, hydrate, solvate, acid salt hydrate, N-oxide, or isomorphic crystalline salt thereof includes a low dose of about 0.2-0.3 mg/kg of body weight to a high dose of about 2-3 mg/kg of body weight.

In one embodiment, the method further includes co-administering an effective amount of one or more of T4, T3, T4AM, rT3, rT3AM, 3,3′-T2, 3,3′-T2AM, 3,5-T2AM, T1, T1AM, T0, and T0AM, wherein the effective amount comprises a daily dosage of between about 1 mcg and about 5000 mg. In one embodiment, the daily dosage is administered in a fortified food or beverage composition.

### III. Examples

Familial hypercholesterolemia (“FH”) is a genetic disorder characterized by high cholesterol levels, specifically very high LDL levels in the blood and early onset of cardiovascular disease. Most cases of familial hypercholesterolemia are associated with mutations in the LDLr gene; mutations in other genes are rare. Patients who have one abnormal copy (i.e., heterozygotes) of the LDLr gene may have premature cardiovascular disease at the age of 30 to 40. Having two abnormal copies (i.e., homozygotes) may cause severe cardiovascular disease in childhood. Risk of cardiovascular disease is increased further with age and in those who smoke, are overweight or obese, have diabetes, and/or high blood pressure. Heterozygous FH is a common genetic disorder, occurring in 1:500 people in most countries; homozygous FH is much rarer, occurring in about 1 in a million births.

Heterozygous FH is normally treated with statins, bile acid sequestrants or other hypolipidemic agents that lower cholesterol levels. Individuals having less than a threshold level of LDL receptor function (e.g., less than about 10%) do not respond to statins or other currently used cholesterol lowering treatments and may require other treatments, including LDL apheresis (removal of LDL in a method similar to dialysis) and, occasionally, liver transplantation.

Presented below are data illustrating the effect of low and high dose T2 administration on wild-type and mutant mice Low dose animals received a daily oral dosage of T2 of 0.25 mg/kg and high dose animals received a daily oral dosage of T2 of 2.5 mg/kg. This corresponds to a dosage for an 80 kg human of about 20-200 mg/day, or a low dose of about 0.2-0.3 mg/kg of body weight to a high dose of about 2-3 mg/kg of body weight. One will appreciate, however, that some subjects (e.g., subjects having very low LDL receptor function) may receive higher doses, depending on need. likewise, some subjects may receive lower doses, which again depends on need.

Mice in the study were fed a standard lab chow referred to as the “Western Diet.” The overall level of fat and the saturated nature of the fat are representative diets typical in the industrialized west that are linked to risk of cardiovascular disease in humans. The formula is used primarily with genetically manipulated rodent models that are susceptible to high cholesterol and cardiovascular disease. The diet may also be useful in diet-induced obesity, diabetes, and metabolic syndrome models. High cholesterol, obesity, and diabetes are each associated increased risk for cardiovascular disease. The composition of the Western Diet is shown below.

TABLE 1
Western Diet

| Component | g/kg |
|---|---|
| Casein | 195.0 |
| DL-Methionine | 3.0 |
| Sucrose | 341.46 |
| Corn Starch | 150.0 |
| Anhydrous Milkfat | 210.0 |
| Cholesterol | 1.5 |
| Cellulose | 50.0 |
| Mineral Mix, AIN-76 (170915) | 35.0 |
| Calcium Carbonate | 4.0 |
| Vitamin Mix, Teklad (40060) | 10.0 |
| Ethoxyquin, antioxidant | 0.04 |

C57 is a wild-type strain, LDLr+/− are a strain carrying one functional copy of the LDLr gene, and the LDLr0 animals are total LDLr knockouts. The LDLr gene encodes for the LDL receptor protein, which is responsible for scavenging LDL with bound cholesterol from the blood and transporting it into cells. Both heterozygous and homozygous LDLr knockout animals develop very high serum cholesterol levels and are considered to be a good model for heterozygous and homozygous familial hypercholesterolemia in humans.

In the data presented below, low and high dose T2 administration did not affect serum triglyceride levels (Table 2) or weight gain (Tables 3 and 4) in any appreciable way. In contrast, both low and high doses of T2 lowered serum cholesterol levels in all animals (Table 5). Low dose T2 administration lowered cholesterol an average of about 38% in C57 animals and about 26% in LDLr+/− animals. High dose T2 administration lowered cholesterol an average of about 67% in C57 and LDLr+/− animals. Surprisingly, T2 administration was able to lower serum cholesterol levels in LDLr knockout animals—low and high dose T2 administration was able to lower cholesterol by an average of about 46% and about 80%, respectively. Cholesterol level in the LDLr knockout animals are still dangerously high, but the fact that T2 was able to lower cholesterol at all is surprising and unexpected given that statin treatment is known to be ineffective in individuals that have no or very low levels of LDL receptor function. LDLr knockout animals have zero LDL receptor function. In addition, high doses of T2 were able to lower serum glucose levels in all animals as compared to controls (Table 6). High doses of T2 were able to lower blood sugar levels in all genetic groups by an average of about 45-50%.

TABLE 2
Triglycerides

| | Before Diet | | 1 Week Diet | | 1 Week Low Dose | | | |
|---|---|---|---|---|---|---|---|---|
| | Con | T2 | Con | T2 | Vehicle | T2 | Vehicle | T2 |
| | | | | | | | 1 Week High Dose | |
| C57 | 51.371 | 83.487 | 53.403 | 224.600 | 19.997 | 13.299 | 20.735 | 45.486 |
| C57 | 69.328 | 134.597 | 183.750 | 228.000 | 34.882 | 36.371 | 43.269 | 50.657 |
| C57 | 60.695 | 72.436 | 217.790 | 54.465 | 32.277 | 43.441 | 53.982 | 72.822 |
| C57 | 77.616 | 68.292 | 173.530 | 176.940 | — | 24.835 | — | 42.161 |
| C57 | 75.544 | 76.235 | 180.340 | 200.770 | 36.371 | — | 41.791 | — |
| C57 | 68.292 | 67.947 | 132.680 | 193.960 | 18.509 | 29.673 | 48.441 | 68.02 |
| Ave | 67.1 | 83.8 | 156.9 | 179.8 | 28.4 | 29.5 | 41.6 | 55.8 |
| Stdev | 9.76 | 25.53 | 57.54 | 64.32 | 8.50 | 11.47 | 12.63 | 13.77 |
| P-value | | | | | | 0.865478 | | 0.127976 |
| LDLr0 | 80.379 | 91.43 | 388.010 | 428.860 | 178.892 | — | 214.677 | — |
| LDLr0 | 100.754 | 94.883 | 296.090 | 313.110 | 227.639 | 230.244 | 162.959 | 173.672 |
| LDLr0 | 81.76 | 93.847 | 394.810 | 377.790 | 189.683 | 106.329 | 167.392 | — |
| LDLr0 | | 79.688 | | 323.320 | | 212.382 | | 210.614 |
| Ave | 87.6 | 90.0 | 359.6 | 360.8 | 198.7 | 183.0 | 181.7 | 192.1 |
| Stdev | 11.39 | 7.00 | 55.14 | 53.54 | 25.60 | 66.98 | 28.67 | 26.12 |
| P-value | | | | | | 0.722927 | | 0.708139 |
| LDLr+/− | 62.767 | 79.688 | 166.720 | 159.920 | 13.671 | 12.927 | 36.25 | 40.683 |
| LDLr+/− | 62.422 | 60.004 | 146.300 | 187.150 | 28.556 | 34.138 | 28.862 | 35.511 |
| LDLr+/− | 63.112 | 71.746 | 146.300 | 170.130 | 25.951 | 40.464 | 38.097 | 62.848 |
| LDLr+/− | 81.76 | 65.875 | 228.000 | 139.490 | 21.486 | 29.673 | 41.791 | 68.389 |
| LDLr+/− | 82.451 | 53.443 | 245.020 | 125.870 | 30.045 | 31.161 | 78.733 | 37.359 |
| LDLr+/− | 64.839 | 80.379 | 47.031 | 210.980 | — | 40.092 | — | 68.02 |
| Ave | 69.6 | 68.5 | 163.2 | 165.6 | 23.9 | 31.4 | 44.7 | 52.1 |
| Stdev | 9.76 | 10.79 | 70.67 | 31.10 | 6.60 | 10.09 | 19.57 | 15.86 |
| P-value | | | | | | 0.260471 | | 0.164619 |
| | | | | | | | 2 Week High Dose | |
| LDLr0 | 83.515 | 54.956 | 252.938 | 217.657 | 288.810 | 183.413 | 239.255 | 225.895 |
| LDLr0 | 80.990 | 83.515 | 263.964 | 282.235 | 384.370 | 260.001 | 327.764 | 274.993 |
| LDLr0 | 67.779 | 82.350 | 264.279 | 261.759 | 270.190 | 239.273 | 302.381 | 402.246 |
| LDLr0 | 141.216 | 86.041 | 321.926 | 226.792 | 304.620 | 268.082 | 372.520 | 281.673 |
| LDLr0 | 111.103 | 99.058 | 272.154 | 281.920 | 345.373 | 244.895 | 418.945 | 462.031 |
| LDLr0 | 64.864 | 127.811 | 366.028 | 265.224 | 222.059 | 177.441 | 369.848 | 223.557 |
| LDLr0 | 89.732 | 83.904 | 325.076 | 276.249 | 317.267 | 263.163 | 490.421 | 485.745 |
| LDLr0 | | 77.881 | | 277.824 | | 252.975 | | 496.099 |
| Ave | 91.3 | 86.9 | 295.2 | 261.2 | 304.7 | 236.2 | 360.2 | 356.5 |
| Stdev | 26.7869 | 20.5814 | 42.5685 | 25.2729 | 52.3331 | 35.6881 | 81.31847 | 117.3491 |
| P-value | | | | | | 0.010278 | | 0.946355 |

TABLE 3
Body weight of Mice on Low Dose T2

| Genotype | Treatment | Baseline | 1 Wk Diet | Day 1 | Day 2 | Day 3 | Day 4 | Day 5 | Day 6 | Day 7 |
|---|---|---|---|---|---|---|---|---|---|---|
| C57 | Vehicle | 28.0 | 31.0 | 30.5 | 31.1 | 30.8 | 29.5 | 30.4 | 30.9 | 31.9 |
| C57 | Vehicle | 26.0 | 31.0 | 29.8 | 30.3 | 30.6 | 29.8 | 30.5 | 30.8 | 31.5 |
| C57 | Vehicle | 25.0 | 28.0 | 27.7 | 29.3 | 29.6 | 28.4 | 29.2 | 29.8 | 29.8 |
| C57 | Vehicle | 25.0 | 28.0 | 27.1 | 28.4 | 28.5 | 27.8 | 26.0 | — | — |
| C57 | Vehicle | 25.0 | 28.0 | 26.1 | 27.2 | 27.9 | 26.4 | 26.9 | 26.7 | 26.7 |
| C57 | Vehicle | 23.0 | 25.0 | 23.8 | 25.5 | 25.4 | 24.9 | 24.9 | 24.4 | 24.4 |
| | Ave | 25.3 | 28.5 | 27.5 | 28.6 | 28.8 | 27.8 | 28.0 | 28.5 | 28.9 |
| | Stdev | 1.63 | 2.26 | 2.46 | 2.06 | 2.02 | 1.88 | 2.38 | 2.86 | 3.23 |
| C57 | T2 | 26.0 | 27.0 | 26.5 | 28.2 | 28.3 | 26.8 | 27.3 | 28.1 | 29.5 |
| C57 | T2 | 24.0 | 28.0 | 27.0 | 27.7 | 27.8 | 27.2 | 28.3 | 29.9 | 30.7 |
| C57 | T2 | 23.0 | 24.0 | 23.0 | 24.1 | 23.3 | 23.7 | 24.5 | 24.8 | 26.2 |
| C57 | T2 | 24.0 | 28.0 | 26.4 | 26.3 | 27.9 | 26.7 | 27.5 | 28.7 | 29.0 |
| C57 | T2 | 24.0 | 24.0 | 24.0 | 25.5 | 26.2 | 25.1 | — | — | — |
| C57 | T2 | 23.0 | 26.0 | 24.1 | 24.9 | 25.6 | 25.4 | 26.4 | 26.1 | 26.1 |
| | Ave | 24.0 | 26.2 | 25.2 | 26.1 | 26.5 | 25.8 | 26.8 | 27.5 | 28.3 |
| | Stdev | 1.10 | 1.83 | 1.66 | 1.60 | 1.90 | 1.33 | 1.45 | 2.05 | 2.06 |
| LDLr0 | Vehicle | 28.0 | 30.0 | 30.0 | 31.3 | 31.2 | 30.3 | 31.2 | 31.5 | 31.7 |
| LDLr0 | Vehicle | 26.0 | 28.0 | 26.6 | 28.1 | 28.5 | 27.8 | 28.8 | 29.4 | 29.4 |
| LDLr0 | Vehicle | 23.0 | 25.0 | 24.3 | 24.9 | 26.4 | 24.7 | 24.5 | 24.6 | 24.8 |
| | Ave | 25.7 | 27.7 | 27.0 | 28.1 | 28.7 | 27.6 | 28.2 | 28.5 | 28.6 |
| | Stdev | 2.52 | 2.52 | 2.87 | 3.20 | 2.41 | 2.81 | 3.39 | 3.54 | 3.51 |
| LDLr0 | T2 | 22.0 | 24.0 | 23.1 | — | — | — | — | — | — |
| LDLr0 | T2 | 20.0 | 23.0 | 21.1 | 21.6 | 22.3 | 21.2 | 22.0 | 22.1 | 22.4 |
| LDLr0 | T2 | 22.0 | 23.0 | 22.7 | 23.6 | 24.4 | 22.9 | 22.2 | 21.7 | 20.6 |
| LDLr0 | T2 | 21.0 | 23.0 | 22.1 | 23.2 | 23.5 | 22.8 | 23.0 | 23.1 | 23.6 |
| | Ave | 21.3 | 23.3 | 22.3 | 22.8 | 23.4 | 22.3 | 22.4 | 22.3 | 22.2 |
| | Stdev | 0.96 | 0.50 | 0.87 | 1.06 | 1.05 | 0.95 | 0.53 | 0.72 | 1.51 |
| LDLr+/− | Vehicle | 23.0 | 24.0 | 23.3 | 24.5 | 24.7 | 23.5 | 23.9 | 24.4 | 25.2 |
| LDLr+/− | Vehicle | 24.0 | 25.0 | 25.2 | 25.9 | 26.4 | 25.1 | 25.7 | 26.1 | 27.3 |
| LDLr+/− | Vehicle | 24.0 | 26.0 | 25.4 | 25.7 | 26.3 | 25.5 | 26.7 | 27.5 | 28.2 |
| LDLr+/− | Vehicle | 25.0 | 26.0 | 26.1 | 27.3 | 27.8 | 26.9 | 27.6 | 27.0 | 27.1 |
| LDLr+/− | Vehicle | 28.0 | 32.0 | 31.3 | 32.9 | 32.4 | 30.8 | 31.6 | 31.5 | 32.1 |
| LDLr+/− | Vehicle | 27.0 | 31.0 | 29.0 | 28.4 | 26.9 | — | — | — | — |
| | Ave | 25.2 | 27.3 | 26.7 | 27.5 | 27.4 | 26.4 | 27.1 | 27.3 | 28.0 |
| | Stdev | 1.94 | 3.33 | 2.91 | 2.99 | 2.64 | 2.76 | 2.87 | 2.63 | 2.55 |
| LDLr+/− | T2 | 27.0 | 28.0 | 28.2 | 29.4 | 29.7 | 28.8 | 29.5 | 30.7 | 31.0 |
| LDLr+/− | T2 | 27.0 | 29.0 | 29.0 | 30.6 | 31.4 | 30.3 | 31.3 | 32.4 | 32.7 |
| LDLr+/− | T2 | 23.0 | 25.0 | 24.7 | 26.6 | 27.2 | 26.2 | 27.3 | 28.5 | 29.6 |
| LDLr+/− | T2 | 25.0 | 27.0 | 26.4 | 27.8 | 28.1 | 27.4 | 28.4 | 29.1 | 29.6 |
| LDLr+/− | T2 | 24.0 | 26.0 | 25.0 | 26.1 | 26.3 | 25.8 | 26.6 | 26.7 | 28.2 |
| LDLr+/− | T2 | 25.0 | 28.0 | 26.6 | 28.2 | 28.8 | 27.3 | 27.9 | 27.9 | 29.0 |
| | Ave | 25.2 | 27.2 | 26.7 | 28.1 | 28.6 | 27.6 | 28.5 | 29.2 | 30.0 |
| | Stdev | 1.60 | 1.47 | 1.70 | 1.69 | 1.82 | 1.68 | 1.69 | 2.05 | 1.60 |

TABLE 4
Body weight of Mice on High Dose T2

| Genotype | Treatment | Baseline | 1 Wk Diet | Day 1 | Day 2 | Day 3 | Day 4 | Day 5 |
|---|---|---|---|---|---|---|---|---|
| C57 | Vehicle | 28.0 | 31.0 | 32.4 | 32.5 | 32.6 | 31.1 | 31.5 |
| C57 | Vehicle | 26.0 | 31.0 | 32.1 | 32.0 | 32.1 | 30.9 | 31.3 |
| C57 | Vehicle | 25.0 | 28.0 | 30.2 | 30.3 | 30.3 | 29.4 | 29.3 |
| C57 | Vehicle | 25.0 | 28.0 | — | — | — | — | — |
| C57 | Vehicle | 25.0 | 28.0 | 28.3 | 28.6 | 28.5 | 27.8 | 28.2 |
| C57 | Vehicle | 23.0 | 25.0 | 25.4 | 25.4 | 25.4 | 25.2 | 24.8 |
| | Ave | 25.3 | 28.5 | 29.7 | 29.8 | 29.8 | 28.9 | 29.0 |
| | Stdev | 1.63 | 2.26 | 2.90 | 2.88 | 2.93 | 2.45 | 2.73 |
| C57 | T2 | 26.0 | 27.0 | 29.6 | 29.5 | 29.8 | 29.1 | 29.7 |
| C57 | T2 | 24.0 | 28.0 | 29.6 | 30.1 | 30.0 | 28.7 | 29.1 |
| C57 | T2 | 23.0 | 24.0 | 24.9 | 25.8 | 25.1 | 23.4 | 24.1 |
| C57 | T2 | 24.0 | 28.0 | 29.8 | 29.7 | 29.9 | 28.8 | 28.5 |
| C57 | T2 | 24.0 | 24.0 | — | — | — | — | — |
| C57 | T2 | 23.0 | 26.0 | 26.7 | 27.1 | 27.2 | 27 | 27.3 |
| | Ave | 24.0 | 26.2 | 28.1 | 28.4 | 28.4 | 27.4 | 27.7 |
| | Stdev | 1.10 | 1.83 | 2.21 | 1.89 | 2.19 | 2.38 | 2.22 |
| LDLr0 | Vehicle | 28.0 | 30.0 | 32.4 | 31.8 | 32.1 | 31.2 | 31.8 |
| LDLr0 | Vehicle | 26.0 | 28.0 | 29.8 | 29.3 | 29.3 | 29.0 | 29.3 |
| LDLr0 | Vehicle | 23.0 | 25.0 | 23.6 | 23.7 | 23.5 | 23.9 | 23.8 |
| | Ave | 25.7 | 27.7 | 28.6 | 28.3 | 28.3 | 28.0 | 28.3 |
| | Stdev | 2.52 | 2.52 | 4.52 | 4.15 | 4.39 | 3.74 | 4.09 |
| LDLr0 | T2 | 22.0 | 24.0 | — | — | — | — | — |
| LDLr0 | T2 | 20.0 | 23.0 | 23.9 | 23.1 | 22.8 | 23.5 | 23.1 |
| LDLr0 | T2 | 22.0 | 23.0 | — | — | — | — | — |
| LDLr0 | T2 | 21.0 | 23.0 | 27.4 | 24.8 | 24.9 | 24.8 | 25.3 |
| | Ave | 21.3 | 23.3 | 25.7 | 24.0 | 23.9 | 24.2 | 24.2 |
| | Stdev | 0.96 | 0.50 | 2.47 | 1.20 | 1.48 | 0.92 | 1.56 |
| LDLr+/− | Vehicle | 23.0 | 24.0 | 24.9 | 24.7 | 25.4 | 23.9 | 24.4 |
| LDLr+/− | Vehicle | 24.0 | 25.0 | 25.5 | 25.4 | 25.6 | 24.4 | 24.7 |
| LDLr+/− | Vehicle | 24.0 | 26.0 | 28.5 | 28.6 | 27.9 | 27.0 | 26.7 |
| LDLr+/− | Vehicle | 25.0 | 26.0 | 27.0 | 26.9 | 26.6 | 25.9 | 26.3 |
| LDLr+/− | Vehicle | 28.0 | 32.0 | 29.6 | 29.8 | 30.0 | 27.7 | 26.0 |
| LDLr+/− | Vehicle | 27.0 | 31.0 | — | — | — | — | — |
| | Ave | 25.2 | 27.3 | 27.1 | 27.1 | 27.1 | 25.8 | 25.6 |
| | Stdev | 1.94 | 3.33 | 1.98 | 2.13 | 1.90 | 1.63 | 1.01 |
| LDLr+/− | T2 | 27.0 | 28.0 | 32.8 | 33.3 | 33.1 | 32.1 | 32.3 |
| LDLr+/− | T2 | 27.0 | 29.0 | 31.8 | 34.5 | 33.9 | 33.0 | 32.9 |
| LDLr+/− | T2 | 23.0 | 25.0 | 29.8 | 29.8 | 29.8 | 29.0 | 28.9 |
| LDLr+/− | T2 | 25.0 | 27.0 | 31.4 | 31.0 | 30.9 | 30.1 | 30.4 |
| LDLr+/− | T2 | 24.0 | 26.0 | 29.2 | 29.1 | 29.3 | 27.9 | 27.6 |
| LDLr+/− | T2 | 25.0 | 28.0 | 29.6 | 29.9 | 29.8 | 26.8 | 26.3 |
| | Ave | 25.2 | 27.2 | 30.8 | 31.3 | 31.1 | 29.8 | 29.7 |
| | Stdev | 1.60 | 1.47 | 1.44 | 2.16 | 1.92 | 2.40 | 2.61 |

TABLE 5
Cholesterol

| | Before Diet | | 1 Week Diet | | 1 Week Low Dose | | | |
|---|---|---|---|---|---|---|---|---|
| | Con | T2 | Con | T2 | Vehicle | T2 | Vehicle | T2 |
| | | | | | | | 1 Week High Dose | |
| C57 | 92.072 | 101.333 | 142.251 | 178.120 | 165.859 | 104.366 | 195.259 | 75.711 |
| C57 | 90.013 | 118.313 | 178.784 | 204.690 | 211.429 | 114.798 | 202.026 | 62.741 |
| C57 | 103.906 | 97.217 | 158.857 | 103.061 | 195.507 | 137.858 | 194.695 | 70.636 |
| C57 | 97.732 | 104.421 | 132.952 | 163.507 | — | 96.130 | — | 41.312 |
| C57 | 91.043 | 102.877 | 126.309 | 138.265 | 154.878 | — | 184.545 | — |
| C57 | 93.101 | 94.13 | 115.681 | 141.587 | 182.330 | 114.249 | 151.838 | 53.718 |
| Ave | 94.6 | 103.0 | 142.5 | 154.9 | 182.0 | 113.5 | 185.7 | 60.8 |
| Stdev | 5.27 | 8.38 | 23.05 | 35.31 | 22.61 | 15.66 | 19.92 | 13.72 |
| P-value | | | | | | 0.0005278 | | 0.0000029 |
| LDLr0 | 234.085 | 200.64 | 1381.710 | 994.950 | 1562.256 | — | 2146.77 | — |
| LDLr0 | 230.483 | 183.66 | 1461.340 | 1432.900 | 1644.582 | 1063.848 | 1629.59 | 303.529 |
| LDLr0 | 222.765 | 179.029 | 1194.020 | 1142.830 | 1400.940 | 593.252 | 891.684 | — |
| LDLr0 | | 168.224 | | 674.308 | | 813.532 | | 337.364 |
| Ave | 229.1 | 182.9 | 1345.7 | 1061.2 | 1535.9 | 823.5 | 1556.0 | 320.4 |
| Stdev | 5.78 | 13.49 | 137.25 | 315.65 | 123.94 | 235.46 | 630.77 | 23.92 |
| P-value | | | | | | 0.0097545 | | 0.0841325 |
| LDLr+/− | 117.799 | 117.284 | 214.653 | 188.748 | 245.470 | 185.075 | 264.62 | 84.169 |
| LDLr+/− | 115.74 | 129.119 | 201.368 | 286.391 | 244.921 | 229.548 | 240.936 | 99.395 |
| LDLr+/− | 114.197 | 136.837 | 180.113 | 241.223 | 263.589 | 196.605 | 247.702 | 103.342 |
| LDLr+/− | 126.546 | 134.264 | 229.267 | 259.822 | 285.550 | 160.917 | 224.582 | 102.778 |
| LDLr+/− | 149.186 | 118.313 | 275.763 | 204.690 | 239.431 | 172.447 | 464.243 | 66.124 |
| LDLr+/− | 132.206 | 145.069 | 219.967 | 299.012 | — | 188.370 | — | 107.29 |
| Ave | 125.9 | 130.1 | 220.2 | 246.6 | 255.8 | 188.8 | 288.4 | 93.8 |
| Stdev | 13.33 | 10.87 | 32.13 | 43.90 | 18.97 | 23.58 | 99.33 | 15.77 |
| P-value | | | | | | 0.0006384 | | 0.0010049 |
| | | | | | | | 2 Week Low Dose | |
| LDLr0 | 175.960 | 149.702 | 662.800 | 727.240 | 845.940 | 611.760 | 939.265 | 996.905 |
| LDLr0 | 185.487 | 180.142 | 668.650 | 856.120 | 1438.580 | 750.720 | 1221.960 | 1035.325 |
| LDLr0 | 131.345 | 171.777 | 797.540 | 727.240 | 1028.640 | 813.250 | 1175.300 | 944.755 |
| LDLr0 | 228.708 | 194.549 | 1166.610 | 832.690 | 1626.150 | 1112.010 | 1960.260 | 988.670 |
| LDLr0 | 201.288 | 185.255 | 885.410 | 1055.300 | 1827.640 | 1167.590 | 1726.965 | 1092.965 |
| LDLr0 | 169.686 | 207.795 | 838.540 | 709.660 | 1174.540 | 438.070 | 1496.420 | 524.830 |
| LDLr0 | 190.831 | 177.354 | 1108.020 | 967.420 | 1612.250 | 1000.840 | 1847.730 | 1189.025 |
| LDLr0 | | 173.636 | | 1119.740 | | 1549.720 | | 1441.530 |
| Ave | 183.3 | 180.0 | 875.4 | 874.4 | 1364.8 | 930.5 | 1481.1 | 1026.8 |
| Stdev | 29.9597 | 17.0920 | 197.6447 | 157.7043 | 357.6818 | 352.0734 | 382.742 | 257.4105 |
| P-value | | | | | | 0.034183 | | 0.017125 |

TABLE 6
Blood Sugar

| | Before Diet | | 1 Week Diet | | 1 Week Low Dose | | 1 Week High Dose | |
|---|---|---|---|---|---|---|---|---|
| | Con | T2 | Con | T2 | Vehicle | T2 | Vehicle | T2 |
| C57 | 136 | 202 | 176 | 123 | 173 | 203 | 239 | 158 |
| C57 | 188 | 139 | 199 | 163 | 142 | 186 | 213 | 106 |
| C57 | 123 | 184 | 144 | 175 | 156 | 190 | 177 | 120 |
| C57 | 186 | 137 | 143 | 149 | — | 120 | — | 92 |
| C57 | 136 | 171 | 156 | 182 | 136 | — | 254 | — |
| C57 | 166 | 171 | 165 | 159 | 206 | 151 | 227 | 74 |
| Ave | 155.8 | 167.3 | 163.8 | 158.5 | 162.6 | 170.0 | 222.0 | 110.0 |
| Stdev | 27.97439 | 25.41391 | 21.32995 | 20.95471 | 28.13894 | 33.93376 | 29.3428 | 31.7805 |
| P-value | | | | | | 0.71715 | | 0.00041 |
| LDLr0 | 139 | 91 | 144 | 160 | 139 | — | 196 | — |
| LDLr0 | 150 | 111 | 181 | 111 | 170 | 143 | 164 | 87 |
| LDLr0 | 111 | 108 | 109 | 151 | 118 | 54 | 157 | — |
| LDLr0 | | 119 | | 141 | | 79 | | 101 |
| Ave | 133.3 | 107.3 | 144.7 | 140.8 | 142.3 | 92.0 | 172.3 | 94.0 |
| Stdev | 20.10804 | 11.78629 | 36.00463 | 21.2975 | 26.15977 | 45.90207 | 20.79263 | 9.899495 |
| P-value | | | | | | 0.174267 | | 0.017303 |
| LDLr+/− | 123 | 122 | 227 | 175 | 224 | 202 | 243 | 108 |
| LDLr+/− | 108 | 124 | 180 | 173 | 238 | 146 | 210 | 99 |
| LDLr+/− | 120 | 142 | 176 | 173 | 226 | 235 | 262 | 112 |
| LDLr+/− | 114 | 182 | 152 | 176 | 205 | 171 | 226 | 94 |
| LDLr+/− | 147 | 142 | 188 | 229 | 142 | 242 | 52 | 113 |
| LDLr+/− | 189 | 124 | 189 | 153 | — | 210 | — | 80 |
| Ave | 133.5 | 139.3 | 185.3 | 179.8 | 207.0 | 201.0 | 198.6 | 101.0 |
| Stdev | 30.28366 | 22.82688 | 24.42676 | 25.56886 | 38.20995 | 36.9973 | 84.20689 | 12.7122 |
| P-value | | | | | | 0.797769 | | 0.019686 |

These data show that T2 may be more effective than statin drugs at lowering cholesterol (at best, statins can only lower serum cholesterol levels about 40-50%) and that T2 can decrease serum cholesterol levels in cases where statin treatment would be ineffective or contraindicated due to the side effects associated with statin treatment. In addition, intake of T2 can lower blood sugar levels relative to controls, which suggests that T2 may be an effective diabetes and metabolic syndrome treatment. Moreover, T2 may be combined with other thyroid hormones (e.g., T4 or T3), cholesterol lowering agents (e.g., statins), anti-diabetes agents, anti-hypertensives, anti-coagulants, anti-anginals, anti-arrhythmics, and/or vitamin and mineral compositions in order to augment the effects of known and yet to be discovered therapeutics and to help maintain healthy triglyceride and cholesterol levels, healthy weight, cardiovascular health, healthy sleep patterns, healthy mood, healthy skin, healthy nails, healthy endocrine function, healthy metabolism, healthy neuropsychiatric function, and a number of other markers of associated with general health and well-being. These data suggest that T2 may be effective for safely and effectively mitigating the effects of increased carbohydrate and/or fat intake and/or high cholesterol and hyperglycemia on normal human metabolic activity without disrupting endogenous thyroid function.

These are interesting and promising results because high cholesterol and hyperglycemia (i.e., diabetes mellitus) are known risk factors for cardiovascular disease. These results are also interesting because one active agent (i.e., T2) is conceivably able to address two known risk factors for cardiovascular disease.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A composition, comprising
a first active agent comprising 3,5-L-T2 ((2S)-2-amino-3-[4-(4-hydroxyphenoxy)-3,5-diiodophenyl]propanoic acid) 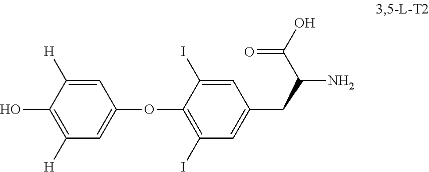
or a prodrug, pharmaceutically acceptable salt, hydrate, solvate, acid salt hydrate, N-oxide, or isomorphic crystalline salt thereof and
a second active agent selected from the group consisting of a thyroid hormone other than 3,5-L-T2, a cholesterol lowering agent, an anti-diabetes agent, an anti-hypertensive, a vasodilator, an inotropic agent, an anti-coagulant, an anti-anginal, an anti-arrhythmic, leptin, leptin analogues, and adipokines, a vitamin and mineral composition, and combinations thereof.

2. The composition of claim 1, further comprising a third active agent selected from the group consisting of T4, T3, T4AM, rT3, rT3AM, 3,3′-T2, 3,3′-T2AM, 3,5-T2AM, T1, T1AM, T0, T0AM, and combinations thereof or a prodrug, pharmaceutically acceptable salt, hydrate, solvate, acid salt hydrate, N-oxide, or isomorphic crystalline salt thereof.

3. The composition of claim 1, wherein the composition is configured to be administered orally, intravenously, transdermally, parenterally, intranasally, by inhalation, by enema, by suppository, topically, and combinations thereof.

4. The composition of claim 1, wherein the cholesterol lowering agent is selected from the group consisting of statins, bile acid sequestrants, nicotinic acid preparations, fibrates, compounds that inhibit digestive absorption of cholesterol, and combinations thereof, wherein the anti-diabetes agent is selected from the group consisting of insulin, sulfonylureas, meglitinides, biguanides, thiazolidinediones, alpha-glucosidase inhibitors, peptide, and combinations thereof, wherein the anti-hypertensive agent is selected from the group consisting of ACE inhibitors, angiotensin II receptor antagonists, alpha blockers, beta blockers, mixed alpha/beta blockers, calcium channel blockers, aldosterone receptor antagonists, vasodilators, diuretics, direct renin inhibitors, and combinations thereof, wherein the anti-coagulant is selected from the group consisting of coumadins, heparins, direct thrombin inhibitors, antiplatelet agents, aspirin, and combinations thereof, wherein the anti-anginal is selected from the group consisting of nitrates, beta blockers, calcium channel blockers, and combinations thereof, wherein the anti-arrhythmic is selected from the group consisting of sodium channel blockers, beta blockers, potassium channel blockers, calcium channel blockers, adenosine, digoxin, and combinations thereof, and wherein the vitamin and mineral composition includes one or more of vitamins A, B₁, B₂, B₃, B₅, B₆, B₇, B₉, B₁₂, C, D, E, and K and/or one or more of potassium, chlorine, sodium, calcium, phosphorus, magnesium, zinc, iron, manganese, copper, iodine, selenium, chromium, molybdenum, and combinations thereof.

5. A method for treating hypercholesterolemia in a subject, the method comprising:
identifying a subject having an elevated serum cholesterol level;
administering to the subject a daily dosage between about 1 mcg and about 5000 mg of 3,5-L-T2 ((2S)-2-amino-3-[4-(4-hydroxyphenoxy)-3,5-diiodophenyl]propanoic acid) 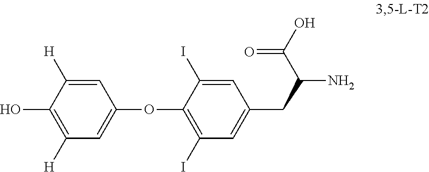
or a prodrug, pharmaceutically acceptable salt, hydrate, solvate, acid salt hydrate, N-oxide, or isomorphic crystalline salt thereof;
obtaining an effect of lowering the subject's serum cholesterol level.

6. The method of claim 5, wherein the daily dosage of 3,5-L-T2 or a prodrug, pharmaceutically acceptable salt, hydrate, solvate, acid salt hydrate, N-oxide, or isomorphic crystalline salt thereof includes a low dose of about 0.2-0.3 mg/kg of body weight to a high dose of about 2-3 mg/kg of body weight.

7. The method of claim 5, wherein the subject is a human.

8. The method of claim 5, wherein the subject is unsuited for statin treatment.

9. The method of claim 5, wherein the subject exhibits reduced LDL receptor function.

10. The method of claim 9, wherein the subject exhibits an LDL receptor function in a range of about 50% of wild-type to less than about 1% of wild-type.

11. The method of claim 5, wherein the subject suffers from one or more deleterious side-effects associated with statin treatment.

12. The method of claim 11, wherein the side-effects associated with statin treatment include muscle pain, muscle weakness, muscle tenderness, myositis, myopathy, rhabdomyolysis, neuropathy, memory loss, changes in liver function, liver failure, changes in kidney function, kidney failure, and combinations thereof.

13. The method of claim 5, wherein 3,5-L-T2 lowers serum cholesterol levels in the subject via a mechanism that is independent of LDL receptor function.

14. The method of claim 5, wherein 3,5-L-T2 is capable of lowering serum cholesterol by about 70%.

15. The method of claim 5, further comprising co-administration of an agent selected from the group consisting of T4, T3, T4AM, rT3, rT3AM, 3,3′-T2, 3,3′-T2AM, 3,5-T2AM, T1, T1AM, T0, T0AM, and combinations thereof or a prodrug, pharmaceutically acceptable salt, hydrate, solvate, acid salt hydrate, N-oxide, or isomorphic crystalline salt thereof.

16. The method of claim 5, further comprising co-administration of a cholesterol lowering agent selected from the group consisting of bile acid sequestrants, nicotinic acid preparations, fibrates, compounds that inhibit digestive absorption of cholesterol, and combinations thereof.

17. A method for treating at least one of metabolic syndrome, hypothyroidism, or thyroid suppression, comprising:
administering to a human a daily dosage of between about 1 mcg and about 5000 mg of 3,5-L-T2 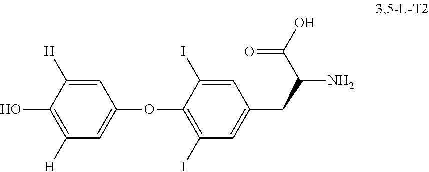
or a prodrug, pharmaceutically acceptable salt, hydrate, solvate, acid salt hydrate, N-oxide, or isomorphic crystalline salt thereof; and
obtaining an effect of establishing or maintaining a healthy metabolism and/or establishing or maintaining healthy endocrine function.

18. The method of claim 17, wherein the daily dosage of 3,5-L-T2 or a prodrug, pharmaceutically acceptable salt, hydrate, solvate, acid salt hydrate, N-oxide, or isomorphic crystalline salt thereof includes a low dose of about 0.2-0.3 mg/kg of body weight to a high dose of about 2-3 mg/kg of body weight.

19. The method of claim 17, further comprising co-administering an effective amount of one or more of T4, T3, T4AM, rT3, rT3AM, 3,3′-T2, 3,3′-T2AM, 3,5-T2AM, T1, T1AM, T0, and T0AM, wherein the effective amount comprises a daily dosage of between about 1 mcg and about 5000 mg.

20. The method of claim 17, wherein the daily dosage is administered in a fortified food or beverage composition.
